(19) United States
(12) Patent Application Publication
(10) Pub. No.: US 2023/0054102 A1
(43) Pub. Date: Feb. 23, 2023

(54) LIQUID- AND SWAB-BASED COLLECTION DEVICES FOR COLLECTING AND PRESERVING BIOLOGICAL MATERIAL

(71) Applicant: Deborah Ann Payne, Lakewood, CO (US)

(72) Inventor: Deborah Ann Payne, Lakewood, CO (US)

(21) Appl. No.: 17/403,893

(22) Filed: Aug. 17, 2021

(51) Int. Cl. A61F 13/38; A61B 10/00; A61F 13/84

(52) U.S. Cl. CPC A61F 13/38; A61B 10/0051; A61F 13/8405

(57) ABSTRACT

A collection device for collecting and preserving biological material. A longitudinal shaft is provided with a proximal end and a distal end. An absorbent solid matrix having at least one layer of chemically impregnated filter paper is affixed to the distal end of the shaft. The absorbent solid matrix consists of chemically impregnated filter paper and can have portions separated by perforations, or an inner core and a separable, overlapping outer layer. The absorbent solid matrix has a configuration that may include nubs, individual ribs, helical, filaments, and radial protrusions. Also disclosed is a tampon-like collection device for delivering an absorbent solid matrix.

### RELATED PATENT APPLICATION

This patent application is related to U.S. provisional patent application No. 63/19,812 for LIQUID-AND SWAB-BASED COLLECTION DEVICES FOR COLLECTING AND PRESERVING BIOLOGICAL MATERIAL, filed Jan. 21, 2021, and hereby incorporates the disclosure thereof in its entirety.

### FIELD OF THE INVENTION

The present invention relates to biological sample collection devices and, more specifically, to a single use liquid- and swab-based collection device that includes an absorbent solid matrix (ASM) for collecting (sampling), processing and preserving (e.g., storing and/or refrigerating) dried body fluid, discharge, and tissue specimens.

### BACKGROUND OF THE INVENTION

The collection, preservation and storage of body fluid, discharge and/or tissue samples for molecular analysis is essential for wellness, infectious disease, and cancer treatment and for research and development of body fluid, discharge and tissue-based biomarkers for disease pathophysiology. Much effort is currently focused on determining markers based on nucleotides (i.e. DNA, mRNA, miRNA), proteins and metabolites for wellness, infectious diseases, cancer staging, prognosis and treatment selection. Detection of these biomarkers would more efficiently direct test recipients and patients to treatments with the highest potential for benefit.

Current tissue preservation methods such as formalin-fixed, paraffin embedded (FFPE) are suitable for histopathology studies but not amenable to biomarker analysis due to poor protein and nucleic acid recovery. Even though recent reports describe some success with genetic analysis, the poor quality of DNA and RNA restricts analysis by common techniques such as RT-PCR, microarrays and sequencing. Extraction of full-length, non-degraded, immunoreactive proteins from FFPE tissue has also proved challenging, with limited detection by common methods such as ELISA and bead-based multiplexed immunoassays. Additionally, exposure of these biomolecules to fixative may chemically alter them.

Another tissue preservation method, snap-freezing of sectioned tissue samples in liquid nitrogen followed by storage at −80° C., has proved more successful for long-term tissue storage of protein and nucleic acid analysis. While this method may be suitable for limited sampling, it is not practical for wide-scale use due to high costs and infrastructure requirements as well as logistical issues in collecting, maintaining and shipping samples at sub-freezing temperatures.

The development of alternative methods for simplified biological sample storage has proved challenging. Meanwhile, methods for blood specimen collection and storage are undergoing a revolution to a new method, known as dried blood spot (DBS) sampling, which offers considerable advantages over traditional venipuncture including decreased costs, reduced sample size and increased analyte stability. More recently, the utility of cards has been demonstrated for the preservation of microbiome samples from stool and viral samples from throat/oral fluid. Once dried, analytes including DNA/RNA, proteins and small molecules are stable at ambient temperature or under refrigeration for years. The detection of miRNA levels were equivalent between wet and dried blood spot. Analytes are extracted from the paper and measured by traditional methods including LC-MS/MS, PCR, RT-PCR, microarray, ELISA, etc.

It would therefore be desirable to have a system that would provide for the collection, and preservation of biological samples in order to enable wide range testing of the samples that are not possible with currently existing methodologies.

### DESCRIPTION OF RELATED ART

U.S. Pat. No. 10,519,434 issued to Morhet, et al. for BIOLOGIC SAMPLE COLLECTION DEVICES AND METHODS OF PRODUCTION AND USE THEREOF, issued on Dec. 31, 2019, discloses collection devices and kits for biological sample collection that include a biologic sample collection device having a hydrophilic swab matrix that includes a modified polycaprolactone (PCL). The biologic sample collection devices, kits and methods are used to collect a biologic sample (e.g., blood, buccal cells, etc.) and to enable extraction of nucleic acids (e.g., DNA) from that biologic sample so that the nucleic acids can be analyzed (e.g., sequencing and subsequent analyses of DNA).

U.S. Pat. No. 5,266,266 issued to Nason for SPECIMEN TEST UNIT, issued on Nov. 30, 1993, discloses a specimen test unit for use in the collection and analysis of a biological specimen or the like. The test unit includes a swab member having a hollow shaft closed at a rear end by a break-off nib, and a swab tip at a front end for collecting a selected specimen. The swab shaft rear end is carried by a housing base, with the break-off nib extending into a reagent chamber having a selected reagent therein. The housing base is sufficiently deformable to break the nib and thereby permit reagent delivery through the swab shaft to contact a collected specimen on the swab tip. In a preferred form, a housing cap removably interfits with the housing base to define a specimen chamber with the swab tip enclosed therein, with the housing base and/or cap being conveniently formed from blow molded plastic components.

U.S. Pat. No. 5,084,005 issued to Kachigian for SWAB FOR COLLECTION OF BIOLOGICAL SAMPLES, issued on Jan. 28, 1992, discloses a swab for collection of biological samples comprising a handle having a proximal end and a distal end and a swabbing tip formed of closed cell polymeric foam at the distal end for contacting and collecting biological samples.

### SUMMARY OF THE INVENTION

In accordance with an embodiment of the invention, there is provided a collection device for collecting and preserving biological material. A longitudinal shaft is provided with a proximal end and a distal end. An absorbent solid matrix (ASM) is affixed to the distal end of the shaft. The absorbent solid matrix consists of chemically impregnated filter paper can have a single portion or multiple portions separated by perforations, or an inner core and a separable, overlapping outer layer. . The absorbent solid matrix has a configuration that may include nubs, individual ribs, helical, filaments, and radial protrusions.

In an alternate embodiment of the invention, a collection device for saliva is provided with a tube or canister having an ASM, but without a shaft or with a shorten staff.

Another embodiment of the invention provides a single layer of two separate filter paper portions optionally separated by perforations, each portion being impregnated with different chemicals.

In still another embodiment of the invention, an insertable swab is encased in a tube and a string is provided for extracting the swab in a manner similar to a tampon.

Yet another embodiment of the invention provides a double layer of overlapping filter papers, the outer layer having apertures for allowing biological material to reach the inner layer, each layer being impregnated with different chemicals.

It is therefore an object of the invention to provide a biological sample collection device. It is a further object of the present invention to provide such a device as a single use liquid- and swab-based collection device.

It is another object of the present invention to provide such a device that includes an absorbent solid matrix for collecting, processing, and preserving body fluid, discharge, and tissue specimens.

It is a further object of the present invention to provide such a collection device with a tampon-like structure to allow an inserted swab to be easily extracted.

It is another object of the present invention to provide the ASM with a configuration that may include nubs, individual ribs, helical, filaments, and radial protrusions.

These and other objects and advantages of the present invention are more readily apparent with reference to the following detailed description and the accompanying drawings.

An embodiment of the invention is directed to a device comprising a swab with absorbent solid matrix (hereinbelow referred to as ASM, which includes FTA® filter paper or other suitable material impregnated with chemicals) on the end thereof, and a container to which the swab can be deposited. Alternatively, the device will be an ASM attached to a string (tampon-like), forming an applicator for the insertion of filter paper or other suitable material, and a container for placing the recovered tampon-like collection device for shipment and storage.

A further embodiment of the invention is directed to a method of extracting and storing a biological sample, the method comprising the steps of: retrieving the collection device from the transport container, processing the collection device so that the biomarkers are eluted from the solid matrix, and analyzing the eluant for detection and/or quantification of biomarkers for surveillance, diagnosis or treatment of diseases.

### BRIEF DESCRIPTION OF THE DRAWINGS

A complete understanding of the present invention may be obtained by reference to the accompanying drawings, when considered in conjunction with the subsequent detailed description, in which:

FIG. 1 is a schematic side view of the collection device in accordance with the invention;

FIGS. 1a-1e are schematic views of alternate swabs for use with various embodiments of the invention;

FIG. 2 is a schematic side view of the collection device showing a filter paper substrate that can be separated from a filter paper substrate sheet;

FIGS. 2a-2b are schematic views of alternate substrates for use with various embodiments of the invention;

FIG. 3 a schematic side view of a break apart embodiment of the collection device;

FIG. 4 a schematic side view of an embodiment of the collection device having an extraction string; and

FIG. 5 is a schematic side view of a tube for collecting saliva.

Like reference numerals refer to like parts throughout the several views of the drawings.

### DETAILED DESCRIPTION OF EXEMPLARY EMBODIMENTS

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

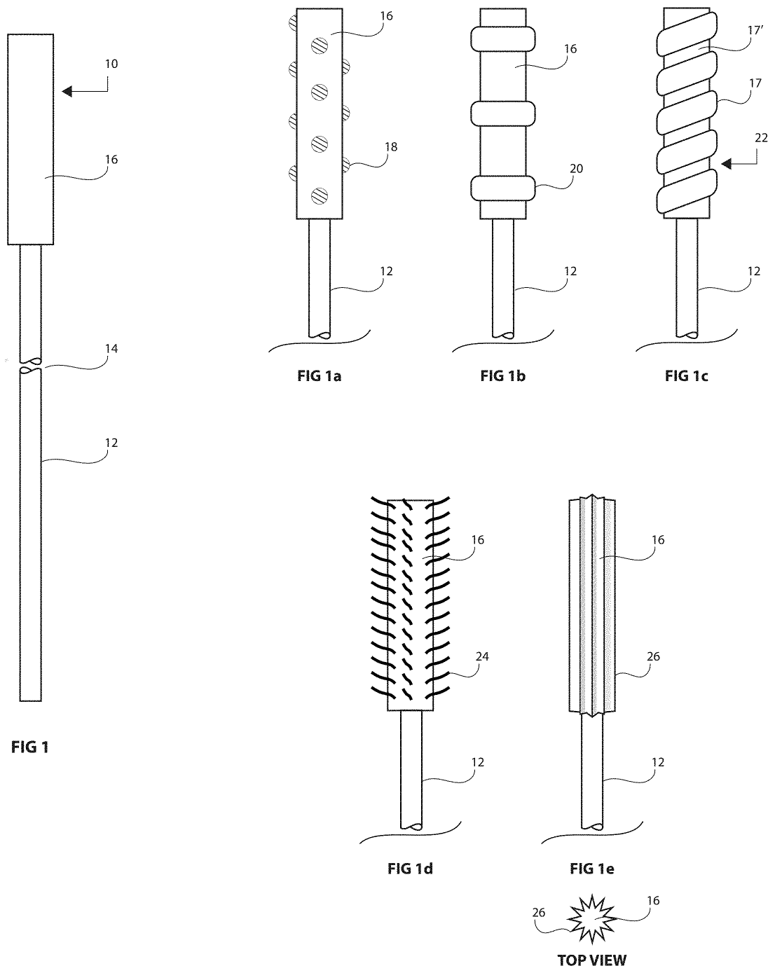

Referring now to FIG. 1, there is shown a schematic side view of the collection device 10 and in accordance with the invention. A longitudinal, cylindrical shaft 12 of paper, plastic, or other suitable material is preferably approximately 4.5″ long and approximately 0.2″ in diameter. Shaft 12 has a weakened or break apart section 14 at a midpoint thereof, so it may be easily separated into two parts to facilitate transportation and storage.

Affixed to the upper portion of shaft 12 is a cylindrical ASM swab 16, being approximately 2″ long and approximately 0.43″ in diameter. Swab 16 is absorbent solid matrix material (ASM) fabricated, for example, from FTA® filter paper under license by Flinders Technologies Pty Ltd., Flinders University, South Australia. Other suitable material can be used, if desired. Such paper or material contains chemicals that lyse cells and inactivate bacteria and viruses while preserving nucleic acids. Samples embedded in FTA cards are suitable for molecular diagnostics.

In operation, ASM swab 16 is inserted into an orifice or a test recipient or used to scrape the surface of the targeted area. The amount of the sample will largely be dependent upon the uses for the sampled body fluid, discharge, and tissue specimen.

Referring now also to FIGS. 1a-1e, various shapes of filter paper 16 can be used for the end collection. Filter paper 16 of swab 16 may be configured to have slight protrusions or nubs 18 (FIG. 1a), ribs 20 (FIG. 1B), candy cane 22 with helical wrapping 17 (FIG. 1c), rigid or flexible filaments or bristles 24 (FIG. 1d), or radial starburst spikes 26 (FIG. 1e) affixed to, or part of the outer surface of ASM material, as shown. In FIG. 1c, it should be understood that helical wrapping 17 and inner core 17′ may be filter papers impregnated with different chemicals, as described infra, or may be an identical filter paper impregnated with identical chemicals. Other configurations of outer coverings for swab 16 are considered within the scope of the invention.

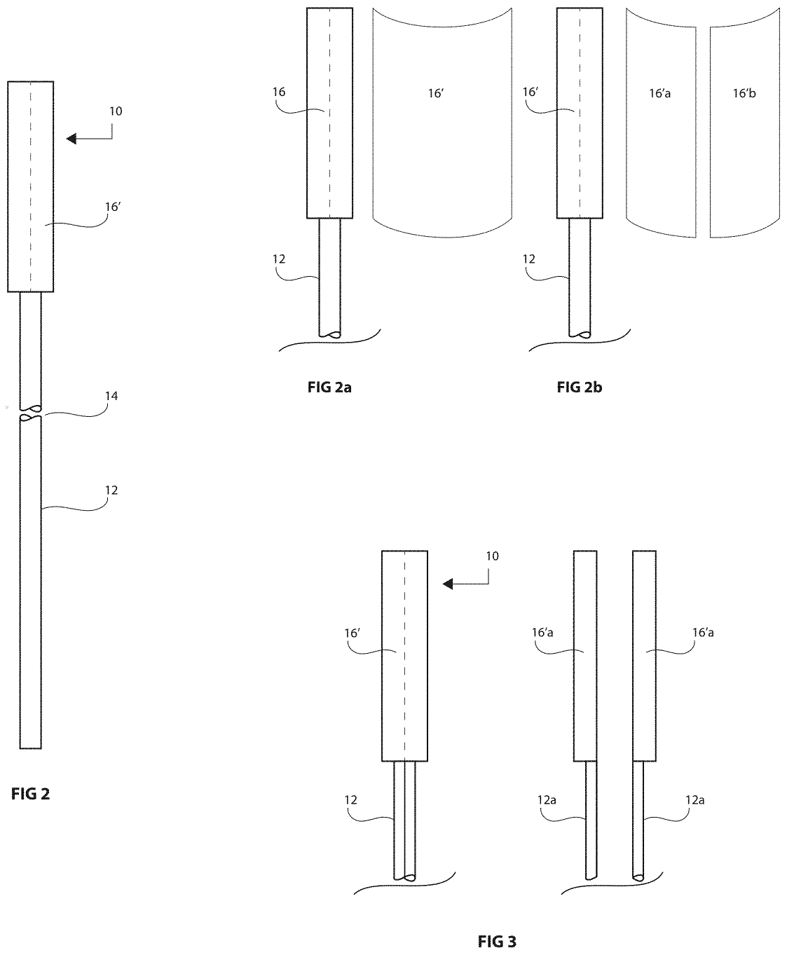

Referring now also to FIGS. 2 and 2a-2b there are shown schematic side views of various embodiments of collection device 10 with an outer filter paper substrate 16′ without or having apertures, not shown, that can be separated from an inner filter paper substrate sheet. Filter paper substrate 16′ can be a unitary sheet or can be divided into two or more portions 16′a, 16′b, with or without perforations, as shown.

ASM swab 16 can be designed to contain different substrates. For example, filter paper substrate 16′a can be optimized for protein collection and can be separated from the filter paper substrate 16′b that may be optimized for nucleic preservation. Additionally, ASM swab 16 may be layered so that an outer layer 16′a can have suitable apertures, not shown, formed therein to allow biological material to pass therethrough and reach inner layer 16′b.

Referring now to FIG. 3, there is shown a schematic side view of a break apart embodiment of collection device 10. Shaft 12 can be separated into two portions 12a, 12b longitudinally when biological samples are to be stored or processed separately.

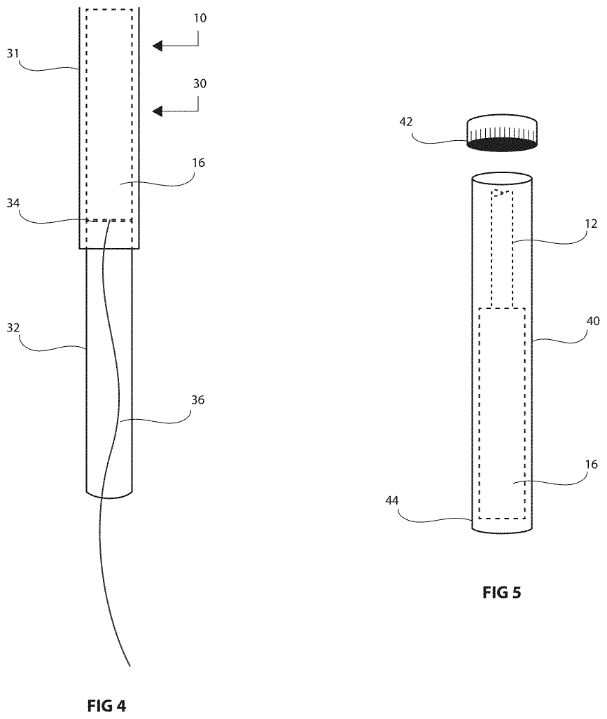

Referring now to FIG. 4 there is shown a schematic side view of an applicator 30 in a tampon-like structure for use with collection device 10. Applicator 30 has a substantially hollow, cylindrical tube 31 being approximately 2.36″ long and with an inner diameter being approximately 0.45″ in the preferred embodiment. ASM swab 16 is positioned in tube 31 and has a diameter no greater than approximately 0.45″. A hand-operated, retractable plunger 32 is provided, being approximately 2.36″ long and having an outside diameter of approximately 0.43″ to slide in tube 31 as is well known in the art. An optional, rigid or semi-rigid plate 34 is proximate the lower portion of swab 16. In operation, plunger 32 is pushed upwardly, expunging swab 16, and applicator 30 is withdrawn from the test recipient. Connected to the lower portion of swab 16 is an extraction string 36 so that swab 16 can be removed from the test recipient when a biological sample is obtained.

Referring now to FIG. 5, there is shown a hollow tube 40 having a replaceable cap 42 at an open end thereof and being closed at the opposite end 44, in which ASM 16 and shaft 12 are disposed. Tube 40 is used to collect saliva as a less intrusive method of obtaining biological material from a subject. The inclusion of a shaft or string, not shown, to extract swab 16 from tube 40 is optional. A shorte shaft 12 is depicted and permits removal of ASM swab 16 if so needed.

Following sample collection, device 10 or portions thereof is placed in a transport container, not shown. ASM swab 16 is then processed for recovery of nucleic acid, protein, and other biomolecules. Various methods and apparatus are available for retrieving biomolecules from the substrate. Some examples of methods include: organic extraction; Chelex 100 resin, such as 142-1253 provided by Bio-Rad Laboratories, Hercules, Calif.; QIAamp™ DNA Investigator Kit, such as 56504 provided by the Qiagen Company, Valencia, Calif.; illustra™ tissue and cells genomicPrep Mini Spin Kit, such as provided by GE Healthcare; and DNA IQ™ Kit, such as TB297 provided by Promega Corp, Madison, Wis.

Biological samples that can be manipulated with the device of the claimed invention include, but are not limited to: biopsied tissue including tumors of liver, skin, kidney, heart, brain, ovaries, and prostate; fecal matter to identify intestinal flora; plant tissue (i.e., food in the form of vegetables, fruits; and crops, trees, and flowers) to identify species, phenotype, viral/bacterial infection; meat to identify species, contamination; forensic tissue to identify DNA; and soil samples; wellness testing.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a great number of variations of the devices, device components, and method steps set forth in the present description. As will be obvious to one of skill in the art, methods, and devices useful for the present methods can include a great number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in any composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The innovations described herein provide ideal preparation and storage of biological specimens with minimal processing and refrigeration, if required, while maintaining sample integrity for biomarker analysis. The inventive collection device 10 using an absorbent solid matrix greatly simplifies biological collection and storage. The invention further provides a simplified, low cost tissue specimen preparation and storage method with minimal processing and refrigeration while maintaining sample integrity for analysis of biomarkers such as mRNA, miRNA, DNA, proteins, and small molecules. Availability of these stable, dried samples allow for simplified analysis of biomarkers to wellness, direct screening, detection, diagnosis, and guiding treatment.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

What is claimed is:

1. A collection device for collecting and preserving biological material, comprising:
a) a substantially longitudinal shaft having a proximal end and a distal end; and
b) an absorbent solid matrix comprising a first layer of filter paper affixed to the distal end of the shaft and extending partially therealong.

2. The collection device for collecting and preserving biological material in accordance with claim 1, wherein the layer of filter paper comprises at least two portions, each portion thereof having paper impregnated with a different chemical.

3. The collection device for collecting and preserving biological material in accordance with claim 2, wherein each portion of the layer of chemically impregnated filter paper is separated from the remaining layers thereof.

4. The collection device for collecting and preserving biological material in accordance with claim 1, wherein the absorbent solid matrix has a configuration chosen from a set of shapes consisting of: nubs, individual ribs, helical, filaments, and radial protrusions.

5. The collection device for collecting and preserving biological material in accordance with claim 1, wherein the filter paper comprises an inner core and a separable, overlapping outer layer, wherein a first of the inner core or outer layer is optimized for protein collection and a second of the inner core or outer layer is optimized for nucleic preservation.

6. The collection device for collecting and preserving biological material in accordance with claim 5, wherein the outer layer of filter paper has a plurality of apertures formed therein for biological material to pass through the outer layer to reach the inner core.

7. The collection device for collecting and preserving biological material in accordance with claim 1, wherein the substantially longitudinal shaft and the absorbent solid matrix are separable longitudinally.

8. The collection device for collecting and preserving biological material in accordance with claim 1, further comprising:
c) a second layer of filter paper proximate the first layer of filter paper; and
d) wherein the first layer of filter paper is impregnated with a first chemical and the second layer of filter paper is impregnated with a second chemical.

9. The collection device for collecting and preserving biological material in accordance with claim 1, further comprising:
c) a tube for collecting saliva, the tube being closed at one end and surrounding the absorbent solid matrix and at least a portion of the substantially longitudinal shaft.

10. A collection device for collecting and preserving biological material, comprising:
a) a substantially longitudinal shaft having a proximal end and a distal end and a weakened portion at a midpoint thereof for breaking the shaft into two sections; and
b) an absorbent solid matrix comprising a layer of chemically impregnated filter paper affixed to the distal end of the shaft and extending partially therealong.

11. The collection device for collecting and preserving biological material in Accordance with claim 10, wherein the layer of filter paper comprises at least two portions, each portion thereof having paper impregnated with a different chemical.

12. The collection device for collecting and preserving biological material in accordance with claim 11, wherein each portion of the layer of chemically impregnated filter paper is separated from the remaining layers thereof.

13. The collection device for collecting and preserving biological material in accordance with claim 10, wherein the absorbent solid matrix has a configuration chosen from a set of shapes consisting of: nubs, individual ribs, helical, filaments, and radial protrusions.

14. The collection device for collecting and preserving biological material in accordance with claim 10, further comprising:
c) a second layer of filter paper proximate the first layer of filter paper; and
d) wherein the first layer of filter paper is impregnated with a first chemical and the second layer of filter paper is impregnated with a second chemical.

15. The collection device for collecting and preserving biological material in accordance with claim 10, further comprising:
c) a tube for collecting saliva, the tube being closed at one end and surrounding the absorbent solid matrix and at least a portion of the substantially longitudinal shaft.

16. The collection device for collecting and preserving biological material in accordance with claim 10, wherein the impregnated filter paper comprises an inner core and a separable outer layer, wherein a first of the inner core or outer layer is optimized for protein collection and a second of the inner core or outer layer is optimized for nucleic preservation.

17. An applicator for a collection device for collecting and preserving biological material, comprising:
a) a substantially hollow, cylindrical tube having an inner circumference having a predetermined dimension and having a proximal aperture;
b) an absorbent solid matrix having an outer circumference dimension smaller than the cylindrical tube inner circumference dimension and having distal border and a proximal border; and
c) a string affixed to the proximal border of the absorbent solid matrix for the removal thereof after the absorbent solid matrix has been inserted into the human cavity.

18. The applicator for a collection device for collecting and preserving biological material in accordance with claim 17, further comprising:
d) a retractable plunger having an outer circumference dimension smaller than the cylindrical tube inner circumference dimension, the plunger being disposed in the aperture of the cylindrical tube for urging the absorbent solid matrix into a human cavity; and
e) a substantially rigid plate proximate and affixed to the proximal border of the absorbent solid matrix, the substantially rigid plate having an aperture therein.

19. The applicator for a collection device for collecting and preserving biological material in accordance with claim 17, wherein the absorbent solid matrix comprises at least two portions of filter paper, each portion thereof having paper impregnated with a different chemical.

20. The applicator for a collection device for collecting and preserving biological material in accordance with claim 17, wherein the impregnated filter paper comprises an inner core and a separable outer layer, wherein a first of the inner core or outer layer is optimized for protein collection and a second of the inner core or outer layer is optimized for nucleic preservation.
